(19) United States
(12) Patent Application Publication
(10) Pub. No.: US 2003/0215526 A1
(43) Pub. Date: Nov. 20, 2003

(54) Stable formulations of angiotensin converting enzyme (ACE) inhibitors

(76) Inventors: Scott Stofik, Perkasie, PA (US); Robert Gwozdz, Telford, PA (US); Christopher Pelloni, Hatfield, PA (US); John C. James, Doylestown, PA (US)

Correspondence Address: John P. White, Cooper & Dunham LLP, 1185 Avenue of the Americas, New York, NY, 10036, US

(21) Appl. No.: 10/384,246

(22) Filed: Mar. 7, 2003

Related U.S. Application Data
(60) Provisional application No. 60/362,737, filed on Mar. 8, 2002.

(51) Int. Cl. A61K 31/401; A61K 33/10; A61K 33/00

(52) U.S. Cl. 424/715; 424/686; 424/687; 514/423

(57) ABSTRACT

A stable pharmaceutical composition comprising a) a therapeutically effective amount of an Angiotensin Converting Enzyme (“ACE”) inhibitor which is susceptible to degradation or its salt; b) a greater than stoichiometric amount of an alkali or alkaline earth metal carbonate, relative to the amount of ACE inhibitor or its salt; and c) a pharmaceutically acceptable carrier, including a process for the manufacture of such compositions.

This application claims the benefit of U.S. Provisional Application No. 60/362,737, filed Mar. 8, 2002, the contents of which are hereby incorporated by reference.

Throughout this application various publications are referenced. The disclosures of these publications in their entireties are hereby incorporated by reference into this application in order to more fully describe the state of the art to which this invention pertains.

### BACKGROUND OF THE INVENTION

Angiotensin Converting Enzyme (“ACE”) inhibitors are useful as antihypertensives. ACE inhibitors are formulated as tablets, capsules or elixirs for oral administration or in sterile solutions or suspensions for parenteral administration.

A number of ACE inhibitors as well as chemically related compounds are disclosed in U.S. Pat. Nos. 4,344,949, 4,374,829, and 4,425,355. Some orally active ACE inhibitors are, for example, ramipril, enalapril, captopril, alacepril, benazepril, ceranapril, cilazapril, delapril, fosinopril, imidapril, libenzapril, lisinopril, moexipril, moveltipril, perindopril, quinapril, spirapril, zofenopril, trandolapril, BPL 36378, CS 622, FPL 63547, and S 9650. Other ACE inhibitors are described, for example, in “Pharmacology of Antihypertensive Therapeutics” (Eds. D. Ganten, P. J. Mutrow) Springer Verlag, Berlin 1990, pp. 377-480.

For example, moexipril hydrochloride, shown below,

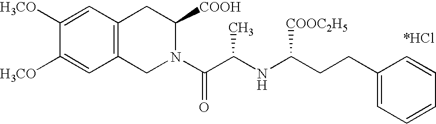

is a prodrug for moexiprilat, which inhibits ACE in humans and animals. The recommended dosage of moexipril is 7.5 to 30 mg daily. However, moexipril, as well as certain other ACE inhibitors and other chemically related compounds, suffer from degradation. When formulated into tablets, degradation of moexipril over time reduces the effective amount of moexipril in the tablet.

The degradation of the ACE inhibitors and chemically related compounds proceeds by any one of, or a combination of, the following routes: 1) hydrolysis of ester groups, particularly the side chain ester groups, 2) oxidation, and internal cyclization which forms various substituted diketopiperazines (“DKPs”). For example, ACE inhibitors which are particularly known to suffer from cyclization are Moexipril, Quinapril, Enalapril and Ramipril; where as Perindopril, Lisinopril, Trandolapril, and Benazepril are also believed to suffer from cyclization. A number of studies on the stability of these compounds have been performed and a number of different stabilization formulations have been developed. See, U.S. Pat. No. 4,743,450, issued May 10, 1988 to Harris et al.; U.S. Pat. No. 5,350,582, issued Sep. 27, 1994 to Merslavic et al.; U.S. Pat. Nos. 5,573,780, issued Nov. 12, 1996, and 5,690,962, Nov. 25, 1997 to Sherman; European Patent No. EP 0 545 194, published Jun. 9, 1993 (KRKA TOVARNA ZDRAVIL (SI)); and Leo Gu, Robert G. Strickley, Li-Hua Chi, Zak T. Chowhan, “Drug-Excipient Incompatability Studies of the Dipeptide Angiotensin-Converting Enzyme Inhibitor, Moexipril Hydrochloride: Dry Powder vs. Wet Granulation”, Pharmaceutical Research, Official Journal of the American Association of Pharmaceutical Scientists, April 1990, Volume 7, Number 4.

U.S. Pat. No. 4,743,450 discloses a composition of the ACE inhibitor Quinapril which is stabilized by magnesium carbonate or a similar alkaline stabilizer in an amount effective to prevent or retard degradation. U.S. Pat. No. 4,743,450 provides no other teaching regarding the amount of the alkaline stabilizer, or any teaching about the process of manufacture of the formulation, or the specific amounts of the ingredients, or the specific characteristics of the ingredients such as particle size, etc.

U.S. Pat. No. 5,350,582 discloses stable solid formulations of enalapril maleate and teaches that a “stoichiometric” amount of a sodium compound should be used to stabilize the enalapril composition. U.S. Pat. No. 5,350,582 provides no teaching regarding the specific amounts of the ingredients, or their particle size.

U.S. Pat. Nos. 5,573,780 and 5,690,962 also relate to stable solid formulations of enalapril and disclose an improved process for the manufacture of such formulations.

### SUMMARY OF THE INVENTION

Disclosed is a pharmaceutical composition comprising a) a therapeutically effective amount of an ACE inhibitor which is susceptible to degradation or its salt, b) a greater than stoichiometric amount of an alkali or alkaline earth metal carbonate, relative to the amount of ACE inhibitor or its salt, and c) a pharmaceutically acceptable carrier.

Also disclosed is a pharmaceutical composition characterized by improved stability comprising a) a therapeutically effective amount of Moexipril.HCl, and b) a greater than stoichiometric amount of sodium bicarbonate, relative to the amount of Moexipril.HCl, so as to form a pharmaceutical composition characterized by improved stability relative to compositions having stoichiometric amounts of sodium bicarbonate or less.

Also disclosed is a process for manufacturing a pharmaceutical solid composition comprising an ACE inhibitor which is susceptible to degradation or its salt, the process comprising:

a) intimately mixing for a predetermined period of time the ACE inhibitor with alkali or alkaline earth metal carbonate;

b) loading the product of step a) into a high shear granulator and mixing for a predetermined period of time with at least one of a pharmaceutically acceptable carrier, a disintegrant, or a binder;

c) granulating the mixture of step b) in the granulator by adding a predetermined amount of water;

d) discharging the granulation of step c) into a holding tank where it is allowed to sit for a predetermined amount of time, after which it is wet milled;

e) drying the product of step d) for a predetermined amount of time and to a predetermined moisture content;

f) further processing the dried material into the solid pharmaceutical composition.

### DETAILED DESCRIPTION OF THE INVENTION

Disclosed is a pharmaceutical composition comprising:

a) a therapeutically effective amount of an ACE inhibitor which is susceptible to degradation or its salt;

b) a greater than stoichiometric amount of an alkali or alkaline earth metal carbonate, relative to the amount of ACE inhibitor or its salt; and

c) a pharmaceutically acceptable carrier.

The degradation may be internal cyclization (DKP formation) The pharmaceutical composition, thus, is characterized by improved stability relative to compositions having stoichiometric amounts of the carbonate or less, wherein the improved stability is based on decreased DKP formation.

In the pharmaceutical composition the alkali or alkaline earth metal carbonate may be intra-granular with the ACE inhibitor. The carbonate may be selected from the group consisting of sodium carbonate, sodium bicarbonate, magnesium carbonate, calcium carbonate, and calcium bicarbonate. Preferably, the carbonate is sodium bicarbonate.

The ACE inhibitor may be selected from the groups consisting of Moexipril, Quinapril, Enalapril, Lisinopril, Perindopril, Ramipril, Trandolapril, and Benazepril. The ACE inhibitor may be Moexipril or Moexipril.HCl. In the pharmaceutical composition the Moexipril.HCl and the sodium bicarbonate may be in intimate contact with each other.

The carbonate may be of a grade wherein 90% of it passes through a 120 micron mesh; or wherein 90% of the carbonate passes through a 105 micron mesh; or wherein 90% of the carbonate passes through a 95 micron mesh.

The pharmaceutical composition may further comprise a disintegrant, a binder, and a lubricant. The pharmaceutically acceptable carrier may be selected from any organic or inorganic substance which is conventionally used in pharmaceutical manufacture, such as a filler, a solvent, or a suspending agent.

In the pharmaceutical composition, the pharmaceutically acceptable carrier may be a filler selected from the group consisting of dibasic calcium phosphate, tribasic calcium phosphate, calcium sulfate, microcrystalline cellulose, powdered cellulose, silicified microcrystalline cellulose, dextrin, dextrose, lactose, magnesium oxide, maltodextrin, maltose, polydextrose, starch, pregelatinized starch, compressible sugar;

the disintegrant may be selected from the groups consisting of Crospovidone NF, alginic acid, carboxymethylcellulose Ca, carboxymethylcellulose Na, croscarmellose Na, guar gum, polacrilin potassium, sodium alginate, and sodium starch glycolate;

the binder may be selected from Pregelatinized Starch NF, acacia, carbomer, carboxymethylcellulose Ca, carboxymethylcellulose Na, corn starch, dextrin, gelatin, guar gum, hydroxypropyl cellulose, hydroxypropyl methylcellulose, polydextrose, povidone, and sodium alginate; and

the lubricant may be selected from the groups consisting of Magnesium Stearate NF, calcium stearate, castor oil, glyceryl monostearate, hydrogenated vegetable oils, polyethylene glycol, sodium stearyl fumarate, stearic acid, talc, and zinc stearate.

The pharmaceutical composition may further comprise a suitable coloring agent and a flavoring agent.

Also disclosed is a pharmaceutical composition characterized by improved stability comprising:

a) a therapeutically effective amount of Moexipril.HCl; and

b) a greater than stoichiometric amount of sodium bicarbonate, relative to the amount of Moexipril.HCl,

so as to form a pharmaceutical composition characterized by improved stability relative to compositions having stoichiometric amounts of sodium bicarbonate or less.

In this pharmaceutical composition the sodium bicarbonate may be intra-granular with the Moexipril.HCl. And, the Moexipril.HCl and the sodium bicarbonate may be in intimate contact with each other.

The carbonate may be of a grade wherein 90% of it passes through a 120 micron mesh; or wherein 90% of the carbonate passes through a 105 micron mesh; or wherein 90% of the carbonate passes through a 95 micron mesh.

Also disclosed is a tablet containing the pharmaceutical compositions described. Also disclosed is a product packaging system comprising the pharmaceutical compositions described in a plastic container bottle, along with a desiccant, which bottle is sealed by a foil that has been attached by heat induction sealing.

In the packaging system, the container bottle may be selected from the group consisting of 40 cc, 150 cc, and 300 cc container bottles; the foil may be selected from the group consisting of 33 mm foil, 38 mm foil, and 53 mm foil; and the desiccant may be a ¾ gram desiccant.

Also disclosed is a process for manufacturing a pharmaceutical solid composition comprising an ACE inhibitor which is susceptible to degradation or its salt, the process comprising:

a) intimately mixing for a predetermined period of time the ACE inhibitor with alkali or alkaline earth metal carbonate;

b) loading the product of step a) into a high shear granulator and mixing for a predetermined period of time with at least one of a pharmaceutically acceptable carrier, a disintegrant, or a binder;

c) granulating the mixture of step b) in the granulator by adding a predetermined amount of water;

d) discharging the granulation of step c) into a holding tank where it is allowed to sit for a predetermined amount of time, after which it is wet milled;

e) drying the product of step d) for a predetermined amount of time and to a predetermined moisture content;

f) further processing the dried material into the solid pharmaceutical composition.

In the process the alkali or alkaline earth metal carbonate, is intra-granular with the ACE inhibitor.

The process may further comprise coating the solid dosage form.

The process may be characterized by resulting in a manufacture of a pharmaceutical composition having an improved stability relative to compositions having stoichiometric amounts of the carbonate or less.

In the process the amount of alkali or alkaline earth metal carbonate in step a) is greater than the stoichiometric amount relative to the amount of ACE inhibitor. The alkali metal carbonate may be sodium bicarbonate.

In the process, the carbonate may be of a grade wherein 90% of it passes through a 120 micron mesh; or wherein 90% of the carbonate passes through a 105 micron mesh; or wherein 90% of the carbonate passes through a 95 micron mesh.

In the process the ACE inhibitor may be Moexipril.HCl.

Step a) of the process may be performed in a blender. The blender may be a tumble blender. The blender may be a tumble V-blender.

The granulation of step c) may be allowed to sit in a holding tank for a predetermined time. The predetermined time may be from about 60 minutes to about 120 minutes, preferably the predetermined time is about 90 minutes.

The process may result in a solid pharmaceutical composition, such as a tablet.

In the process, the amount of alkali or alkaline earth metal carbonate in step a) may be greater than the stoichiometric amount relative to the amount of ACE inhibitor; step a) may be performed in a blender; the alkali metal carbonate may be sodium bicarbonate; and the ACE inhibitor may be Moexipril.HCl, thus resulting in a pharmaceutical composition having an improved stability relative to compositions having stoichiometric amounts of the sodium bicarbonate or less.

This invention will be better understood from the Experimental Details which follow. However, one skilled in the art will readily appreciate that the specific methods and results discussed are merely illustrative of the invention as described more fully in the claims which follow thereafter.

### EXPERIMENTAL DETAILS

Process Overview

In some of the following examples, the Sodium Bicarbonate and Moexipril.HCl are placed in a blender, which may be a tumble blender, more specifically a V-blender, and intimately blended for a period of time. The Intra-granular materials are added to a granulator, such as a high shear granulator, including the Moexipril HCl/Sodium Bicarbonate blend. The materials in the granulator are mixed for a period of time and then granulated for a period of time with a known amount of water added. The wet granulation is discharged, allowed to sit for a predetermined time, e.g. 60-120 minutes, preferably 90 minutes, and then wet milled. The wet milled material is dried in a fluid bed dryer to a predetermined moisture content and discharged. The dried granulation is then milled, and blended with the remainder of the Extra-granular excipients. The resultant final blend is then pressed into tablets. Lastly, they are coated with a water soluble polymer coat and clear coat. The final product is then packaged.

### Example 1—Excess of Sodium Bicarbonate

Batch Nos. 001-003

FORMULA (* indicates tablets that were coated with a water soluble polymer coat; ** indicates non-coated tablets)

| Ingredient | 001* | 002** | 003* |
|---|---|---|---|
| Intra-Granular | | | |
| Moexipril HCl | 15.00 | 15.00 | 15.00 |
| Sodium Bicarbonate | 5.60 | 5.60 | 10.00 |
| Lactose Monohydrate, NF | 155.90 | 150.30 | 141.50 |
| Crospovidone | 6.00 | 6.00 | 6.00 |
| Pregelatinized Starch | 16.00 | 16.00 | 16.00 |
| Extra-Granular | | | |
| Sodium Bicarbonate | — | 5.60 | 10.00 |
| Crospovidone | 4.00 | 4.00 | 4.00 |
| Magnesium Stearate, NF | 1.00 | 1.00 | 1.00 |

Stability (DKP Formation)

| Batch | 4 weeks @ 40° C./75% RH | 12 weeks @ 40° C./75% RH |
|---|---|---|
| 001* | 2.7% | 3.7% |
| 002** | 1.6% | N/T |
| 003* | 0.27% | 0.5% |

The tablets of batch 002 were not coated. However, the cores were placed on stability with the same packaging configuration as the others (HDPE bottle, Foil seal cap, and desiccant). One would expect a slightly higher value for DKP (DiKetoPiperazine) formation due to the added stresses endured on the tablets during coating.

The stability study of batch 002 was stopped after four weeks since the DKP (DiKetoPiperazine) value was higher than desired at this early time point.

These experiments show that as the amount of Sodium Bicarbonate increases, even above the stoichiometric amount, the formulation becomes more stable.

### Example 2—Intimate Blending and Intra-Granular Sodium Bicarb.

PROCESS—An important step that we found to contribute to the stability of Moexipril is the intimate blending of the Sodium Bicarbonate with the Moexipril HCl. This is done just prior to wet granulation. The Moexipril HCl and the Sodium Bicarbonate were placed in a V-Blender together and intimately blended. Once homogeneously mixed, the materials were place in the high shear granulator with the other ingredients and granulated.

Formula

| Ingredient | 004* | 002 |
|---|---|---|
| Intra-Granular | | |
| Moexipril HCl | 15.00 | 15.00 |
| Sodium Bicarbonate | 11.2 | 5.60 |
| Lactose Monohydrate, NF | 150.30 | 150.30 |
| Crospovidone | 6.00 | 6.00 |
| Pregelatinized Starch | 16.00 | 16.00 |
| Extra-Granular | | |
| Sodium Bicarbonate | — | 5.60 |
| Crospovidone | 4.00 | 4.00 |
| Magnesium Stearate, NF | 1.00 | 1.00 |

*Process of batch 004 contained the intimate blending step.

Stability (DKP Formation)

| BATCH | 4 weeks @ 40° C./75% RH |
|---|---|
| 004 | 0.4% |
| 002 | 1.6% |

With the two formulations listed above, the process for batch 004 utilized the extra step of intimate blending and all of the sodium bicarbonate was intra-granular. The stability data indicate that the process of intimate blending and internal sodium bicarbonate made the formulation more stable. The Stability study was stopped at the 4 week time point for the batch 002 due to its higher DKP value (%). The DKP value for the batch 004 formulation remained relatively unchanged for the duration of the study (12 weeks).

### Example 3—Particle Size of Sodium Bicarbonate

FORMULA (All Tablets in this Example were Coated)

| Ingredient | 003 | 005 |
|---|---|---|
| Intra-Granular | | |
| Moexipril HCl | 15.00 | 15.00 |
| Sodium Bicarbonate | 10.00 | — |
| Sodium Bicarbonate, Extra Fine | — | 10.00 |
| Lactose Monohydrate, NF | 141.50 | 141.50 |
| Crospovidone | 6.00 | 6.00 |
| Pregelatinized Starch | 16.00 | 16.00 |
| Extra-Granular | | |
| Sodium Bicarbonate | 10.00 | — |
| Sodium Bicarbonate, Extra Fine | — | 10.00 |
| Crospovidone | 4.00 | 4.00 |
| Magnesium Stearate, NF | 1.00 | 1.00 |

Stability (DKP Formation)

| Batch | 12 weeks @ 40° C./75% RH |
|---|---|
| 003 | 0.5% |
| 005 | 0.4% |

These two experiments differed in the particle size of the sodium bicarbonate used. Batch 003 used the standard grade, whereas batch 005 used the extra-fine grade [d(v, 0.90): 95 micron—i.e. 90% of the material passes through a 95 micron mesh screen]. The data shows improvement of the extra-fine grade over the standard grade. Both batches were coated and placed on stability. Both formulations had the same packaging configurations: HDPE bottle, Foil seal cap, and desiccant packet. Thus, the smaller the particle size is of the sodium bicarbonate, the more stable the resultant formulation.

### Example 4—Final Process and Formulation—Excess Sodium Bicarbonate, Intimate Blending and Greater Than Stoichiometric Amount of Internal Sodium Bicarbonate, Extra Fine

With the improvements, one would expect that if all three were used in conjunction, a suitable and stable formulation would be obtained.

| Ingredient | 008/009 |
|---|---|
| Intra-Granular | |
| Moexipril HCl | 15.00 |
| Sodium Bicarbonate, Extra Fine | 11.20 |
| Lactose Monohydrate, NF | 140.50 |
| Crospovidone | 6.00 |
| Pregelatinized Starch | 16.00 |
| Extra-Granular | |
| Sodium Bicarbonate, Extra Fine | 8.80 |
| Crospovidone | 4.00 |
| Magnesium Stearate, NF | 1.00 |

The Process for both contained the “intimate blending” step with the Moexipril HCl and Sodium Bicarbonate, Extra Fine, as well as a stoichiometric excess of internal sodium bicarbonate. The Tablets were coated with a water soluble polymer coating.

Stability (DKP Formation)

| Batch | 12 weeks @ 40° C./75% RH |
|---|---|
| 008 | 1.0% |
| 009 | 0.9% |

Stability data indicates a suitable, stable formulation. The values of 1.0% and 0.9% for DKP formation are considered acceptable at stress conditions. Though other experimental batches have yielded a slightly lower value, we attribute this slight increase over the 12 weeks at accelerated conditions to batch scale up. Previous batches were manufactured at the pilot scale level. The 008 batch was granulated in two 50 liter sub-batches whereas batch 009 was granulated in ten 50 liter sub-batches. When compared to experimental batch 001, the first scale up batch, which consisted of one 50 liter batch, and had a value of 3.7% DKP formation, it is a great improvement.

Table 1 summarizes the data collected.

TABLE 1

| | | | | extra | | | | | | |
|---|---|---|---|---|---|---|---|---|---|---|
| | | Extra | | fine | | | | | | |
| | | Na- | intimate | part. | initial | 2 | 4 | 6 | 8 | 12 |
| Batch | coated | bicarb | blend | size | DKP | weeks | weeks | weeks | weeks | weeks |
| 001* | yes | no | no | no | 0.4% | NT | 2.7% | 2.4% | 2.9% | 3.7% |
| 002 | no | yes | no | no | 0.3% | 1.0% | 1.6% | NT | NT | NT |
| 003 | yes | yes | no | no | 0.1% | 0.0% | 0.3% | 0.5% | 0.5% | 0.5% |
| 004 | no | yes | yes | no | 0.1% | 0.2% | 0.4% | 0.3% | 0.2% | 0.3% |
| 005 | yes | yes | no | yes | 0.1% | 0.0% | 0.2% | 0.3% | 0.5% | 0.4% |
| 006 | no | no | yes | no | 0.3% | 0.5% | 1.0% | NT | NT | NT |
| 007 | no | Yes | no | no | 0.2% | 0.3% | 0.8% | 0.6% | 1.1% | 1.4% |
| | | x2 | | | | | | | | |
| 008* | yes | yes | yes | yes | 0.3% | 0.3% | 0.4% | 0.5% | 0.9% | 1.0% |
| 009* | yes | yes | yes | yes | 0.2% | NT | 0.5% | NT | 0.6% | 0.9% |

*indicates Scaled-up Batches.

Packaging

To further improve stability and protect against degradation of the formulation, a heat induction seal (“HIS”) liner is used on the container bottles. The desiccant may be in a canister, in a capsule, in a pouch, or in a pre-cut pouch. A ¾ gram desiccant is also used. The HIS liner foil may be, for example, 33 mm, 38 mm, or 53 mm. The container bottles may be 40 cc, 150 cc or 300 cc. Typically, Moexipril HCl formulations may be at 7.5 mg per tablet and 15 mg per tablet. The tablets may be packaged in 100's or 1000's per bottle.

What is claimed is:

1. A pharmaceutical composition comprising:
a) a therapeutically effective amount of an ACE inhibitor which is susceptible to degradation or its salt;
b) a greater than stoichiometric amount of an alkali or alkaline earth metal carbonate, relative to the amount of ACE inhibitor or its salt; and
c) a pharmaceutically acceptable carrier.

2. The pharmaceutical composition of claim 1, wherein the degradation is internal cyclization (DKP formation).

3. The pharmaceutical composition of claim 1, characterized by improved stability relative to compositions having stoichiometric amounts of the carbonate or less, wherein the improved stability is based on decreased DKP formation.

4. The pharmaceutical composition of claim 1, wherein the alkali or alkaline earth metal carbonate is intra-granular with the ACE inhibitor.

5. The pharmaceutical composition of claim 1, wherein the carbonate is selected from the group consisting of sodium carbonate, sodium bicarbonate, magnesium carbonate, calcium carbonate, and calcium bicarbonate.

6. The pharmaceutical composition of claim 1, wherein the ACE inhibitor is Moexipril.

7. The pharmaceutical composition of claim 1, wherein the Moexipril.HCl and the sodium bicarbonate is in intimate contact with each other.

8. The pharmaceutical composition of claim 1, wherein the carbonate is sodium bicarbonate.

9. The pharmaceutical composition of claim 1, wherein 90% of the carbonate passes through a 120 micron mesh.

10. The pharmaceutical composition of claim 1, wherein 90% of the carbonate passes through a 105 micron mesh.

11. The pharmaceutical composition of claim 1, wherein 90% of the carbonate passes through a 95 micron mesh.

12. The pharmaceutical composition of claim 1, wherein the pharmaceutically acceptable carrier is a filler selected from the group consisting of dibasic calcium phosphate, tribasic calcium phosphate, calcium sulfate, microcrystalline cellulose, powdered cellulose, silicified microcrystalline cellulose, dextrin, dextrose, lactose, magnesium oxide, maltodextrin, maltose, polydextrose, starch, pregelatinized starch, compressible sugar.

13. The pharmaceutical composition of claim 1, wherein the ACE inhibitor is selected from the groups consisting of Moexipril, Quinapril, Enalapril, Lisinopril, Perindopril, Ramipril, Trandolapril, and Benazepril.

14. The pharmaceutical composition of claim 1, further comprising a disintegrant, a binder, and a lubricant.

15. The pharmaceutical composition of claim 14,
wherein the pharmaceutically acceptable carrier is a filler selected from the group consisting of dibasic calcium phosphate, tribasic calcium phosphate, calcium sulfate, microcrystalline cellulose, powdered cellulose, silicified microcrystalline cellulose, dextrin, dextrose, lactose, magnesium oxide, maltodextrin, maltose, polydextrose, starch, pregelatinized starch, compressible sugar;
wherein the disintegrant is selected from the groups consisting of Crospovidone NF, alginic acid, carboxymethylcellulose Ca, carboxymethylcellulose Na, croscarmellose Na, guar gum, polacrilin potassium, sodium alginate, and sodium starch glycolate;
wherein the binder is selected from Pregelatinized Starch NF, acacia, carbomer, carboxymethylcellulose Ca, carboxymethylcellulose Na, corn starch, dextrin, gelatin, guar gum, hydroxypropyl cellulose, hydroxypropyl methylcellulose, polydextrose, povidone, and sodium alginate; and
wherein the lubricant is selected from the groups consisting of Magnesium Stearate NF, calcium stearate, castor oil, glyceryl monostearate, hydrogenated vegetable oils, polyethylene glycol, sodium stearyl fumarate, stearic acid, talc, and zinc stearate.

16. The pharmaceutical composition of claim 1, further comprising a suitable coloring agent and a flavoring agent.

17. A pharmaceutical composition characterized by improved stability comprising:
a) a therapeutically effective amount of Moexipril.HCl; and
b) a greater than stoichiometric amount of sodium bicarbonate, relative to the amount of Moexipril.HCl,
so as to form a pharmaceutical composition characterized by improved stability relative to compositions having stoichiometric amounts of sodium bicarbonate or less.

18. The pharmaceutical composition of claim 17, wherein the sodium bicarbonate is intra-granular with the Moexipril.HCl.

19. The pharmaceutical composition of claim 17, wherein 90% of the carbonate passes through a 120 micron mesh.

20. The pharmaceutical composition of claim 17, wherein 90% of the carbonate passes through a 105 micron mesh.

21. The pharmaceutical composition of claim 17, wherein 90% of the carbonate passes through a 95 micron mesh

22. The pharmaceutical composition of claim 17, wherein the Moexipril.HCl and the sodium bicarbonate is in intimate contact with each other.

23. A tablet containing the pharmaceutical composition of claim 1.

24. A tablet containing the pharmaceutical composition of claim 17.

25. A product packaging system comprising the pharmaceutical composition of claim 17 in a plastic container bottle, along with a desiccant, which bottle is sealed by a foil that has been attached by heat induction sealing.

26. The packaging system of claim 25,
wherein the container bottle is selected from the group consisting of 40 cc, 150 cc, and 300 cc container bottles;
wherein the foil is selected from the group consisting of 33 mm foil, 38 mm foil, and 53 mm foil; and
wherein the desiccant is ¾ gram desiccant.

27. A process for manufacturing a pharmaceutical solid composition comprising an ACE inhibitor which is susceptible to degradation or its salt, the process comprising:
a) intimately mixing for a predetermined period of time the ACE inhibitor or its salt with alkali or alkaline earth metal carbonate;
b) loading the product of step a) into a high shear granulator and mixing for a predetermined period of time with at least one of a pharmaceutically acceptable carrier, a disintegrant, or a binder;
c) granulating the mixture of step b) in the granulator by adding a predetermined amount of water;
d) discharging the granulation of step c) into a holding tank where it is allowed to sit for a predetermined amount of time, after which it is wet milled;
e) drying the product of step d) for a predetermined amount of time and to a predetermined moisture content;
f) further processing the dried material into the solid pharmaceutical composition.

28. The process of claim 27, wherein the alkali or alkaline earth metal carbonate is intra-granular with the ACE inhibitor.

29. The process of claim 27, further comprising coating the solid dosage form.

30. The process of claim 27, characterized by the manufacture of a pharmaceutical composition having an improved stability relative to compositions having stoichiometric amounts of the carbonate or less.

31. The process of claim 27, wherein the amount of alkali or alkaline earth metal carbonate in step a) is greater than the stoichiometric amount relative to the amount of ACE inhibitor.

32. The process of claim 27, wherein the alkali metal carbonate is sodium bicarbonate.

33. The process of claim 27, wherein 90% of the carbonate passes through a 120 micron mesh.

34. The process of claim 27, wherein 90% of the carbonate passes through a 105 micron mesh.

35. The process of claim 27, wherein 0.90% of the carbonate passes through a 95 micron mesh.

36. The process of claim 27, wherein the ACE inhibitor is Moexipril-HCl.

37. The process of claim 27, wherein step a) is performed in a blender.

38. The process of claim 37, wherein the blender is a tumble blender.

39. The process of claim 38, wherein the blender is a tumble V-blender.

40. The process of claim 27, the granulation of step c) is allowed to sit in a holding tank for a predetermined time.

41. The process of claim 40, wherein the predetermined time is from about 60 minutes to about 120 minutes.

42. The process of claim 40, wherein the predetermined time is about 90 minutes.

43. The process of claim 27, wherein the solid pharmaceutical composition is a tablet.

44. The process of claim 27,
wherein the amount of alkali or alkaline earth metal carbonate in step a) is greater than the stoichiometric amount relative to the amount of ACE inhibitor;
wherein step a) is performed in a blender;
wherein the alkali metal carbonate is sodium bicarbonate; and
wherein the ACE inhibitor is Moexipril.HCl,
so as to result in a pharmaceutical composition having an improved stability relative to compositions having stoichiometric amounts of the sodium bicarbonate or less.
